(19) United States
(12) Patent Application Publication
(10) Pub. No.: US 2018/0360731 A1
(43) Pub. Date: Dec. 20, 2018

(54) ADHESIVE REMOVER AND METHODS OF USING THE SAME

(71) Applicant: FARAH PRODUCTS LLC, Miami, FL (US)

(72) Inventor: Andrea FARAH, Miami, FL (US)

(21) Appl. No.: 16/013,252

(22) Filed: Jun. 20, 2018

Related U.S. Application Data
(60) Provisional application No. 62/522,404, filed on Jun. 20, 2017.

(51) Int. Cl. A61K 8/92; A61K 8/46

(52) U.S. Cl. CPC A61K 8/922; A61Q 1/14; A61K 8/463

(57) ABSTRACT

Embodiments herein are directed to a composition for removing an adhesive comprising an essential oil and an alkyl sulfate or a salt thereof. In some embodiments, the essential oil is melaleuca oil. The adhesive remover may be used for removing any cosmetic item adhered on to a subject's body part, such as false eyelashes. Embodiments herein also disclose kits and methods of use.

### SUMMARY

Embodiments herein are directed to a composition for removing an adhesive comprising an essential oil and an alkyl sulfate or a salt thereof. In some embodiments, the essential oil is melaleuca oil, eucalyptus oil, Eucalyptus giobuius oil, peppermint oil, lime oil, citrus oil, tea tree oil, juniper oil, almond oil, anise oil, buchu oil, celery oil, cumin oil, nutmeg oil, cassia oil, cinnamon oil, sassafras oil, camphor oil, cedar oil, rosewood oil, sandalwood oil, agarwood oil, galangal oil, ginger oil, basil oil, bay leaf oil, cinnamon oil, sage oil, guava oil, lemongrass oil, oregano oil, patchouli oil, pine oil, rosemary oil, spearmint oil, thyme oil, tsuga oil, wintergreen oil, benzoin oil, copaiba oil, frankincense oil, myrrh oil, cannabis oil, chamomile oil, clary sage oil, clove oil, geranium oil, or any combination thereof. In some embodiments, the oil is melaleuca oil. In some embodiments, the alkyl sulfate or a salt thereof is naturally-derived. In some embodiments, the alkyl sulfate or a salt thereof is a palm-derived alkyl sulfate, a coconut-derived alkyl sulfate, or a combination thereof In some embodiments, the alkyl sulfate is sodium coco sulfate, ammonium lauryl sulfate (ALS), sodium pareth sulfate, sodium lauryl sulfate, sodium laureth sulfate, or a combination thereof.

Some embodiments are directed to methods of removing an adhesive comprising applying onto the adhesive a composition comprising an alkyl sulfate or a salt thereof, and an essential oil. Some embodiments are directed to methods of removing an adhesive comprising applying onto the adhesive a composition comprising an alkyl sulfate or a salt thereof, and melaleuca oil. In some embodiments, the adhesive comprises an acrylate, an acrylamide, a cellulose, or a combination thereof. Some embodiments are directed to a method of removing false eyelashes comprising applying to the false eyelashes a composition comprising an alkyl sulfate or a salt thereof, and an essential oil. Some embodiments are directed to a method of removing false eyelashes comprising applying to the false eyelashes a composition comprising an alkyl sulfate or a salt thereof, and melaleuca oil. Some embodiments are directed to a method of removing a cosmetic item adhered to a body part comprising applying to the body part a composition comprising an alkyl sulfate or a salt thereof, and an essential oil. Some embodiments are directed to a method of removing a cosmetic item adhered to a body part comprising applying to the body part a composition comprising an alkyl sulfate or a salt thereof, and melaleuca oil.

Some embodiments are directed to a kit comprising an adhesive remover comprising an essential oil and an alkyl sulfate or a salt thereof; and instructions for use in methods for removing an adhesive. Some embodiments are directed to a kit comprising an adhesive remover comprising an essential oil and an alkyl sulfate or a salt thereof; and instructions for use in methods for removing a cosmetic item adhered to a body part. Some embodiments are directed to a kit comprising a false eyelash remover comprising an essential oil and an alkyl sulfate or a salt thereof; and instructions for use in methods for removing a false eyelash.

### DETAILED DESCRIPTION

Before the present compositions and methods are described, it is to be understood that this invention is not limited to the particular processes, formulations, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

Embodiments herein are directed to a composition for removing an adhesive comprising an essential oil and a surfactant. Some embodiments herein are directed to a composition for removing an adhesive comprising an essential oil and an alkyl sulfate or a salt thereof. Some embodiments herein are directed to a composition for removing an adhesive comprising melaleuca oil and an alkyl sulfate or a salt thereof. As used herein, the term “removing” shall mean complete removal as well as breaking down, softening or dissolving at least partially of the adhesive.

In some embodiments, the essential oil is melaleuca oil, eucalyptus oil, Eucalyptus giobuius oil, peppermint oil, lime oil, citrus oil, juniper oil, almond oil, anise oil, buchu oil, celery oil, cumin oil, nutmeg oil, cassia oil, cinnamon oil, sassafras oil, camphor oil, cedar oil, rosewood oil, sandalwood oil, agarwood oil, galangal oil, ginger oil, basil oil, bay leaf oil, cinnamon oil, sage oil, guava oil, lemongrass oil, oregano oil, patchouli oil, pine oil, rosemary oil, spearmint oil, thyme oil, tsuga oil, wintergreen oil, benzoin oil, copaiba oil, frankincense oil, myrrh oil, cannabis oil, chamomile oil, clary sage oil, clove oil, geranium oil, or any combination thereof. In some embodiments, the essential oil is melaleuca oil, also known as tea tree oil. In some embodiments, the essential oil is in the composition in an amount of about 2.5% v/v to about 20% v/v. In some embodiments, the essential oil is in an amount of about 2.5% v/v to about 20% v/v, about 5% v/v to about 20% v/v, about 7% v/v to about 20% v/v, about 10% v/v to about 20% v/v, about 15% v/v to about 20% v/v, about 2.5% v/v to about 10% v/v, about 2.5% v/v to about 5% v/v, or any value within these ranges. In some embodiments, the essential oil is in an amount of about 2.5% v/v, about 5% v/v, about 5.5% v/v, about 6% v/v, about 7% v/v, about 7.5% v/v, about 8% v/v, about 9% v/v, about 10% v/v, about 12% v/v, about 15% v/v, about 20% v/v, or a range between any two of these values.

In some embodiments, the alkyl sulfate or a salt thereof is naturally-derived. In some embodiments, the alkyl sulfate or a salt thereof is a palm-derived alkyl sulfate, a coconut-derived alkyl sulfate, or a combination thereof In some embodiments, the alkyl sulfate is sodium coco sulfate, sodium lauryl sulfate, ammonium lauryl sulfate (ALS), sodium pareth sulfate, sodium laureth sulfate, or a combination thereof. In some embodiments, the alkyl sulfate is in an amount of about 0.01% v/v to about 5% v/v, about 0.1% v/v to about 5% v/v, about 0.1% v/v to about 4% v/v, about 0.1% v/v to about 3% v/v, about 0.1% v/v to about 2.5% v/v, about 0.1% v/v to about 2% v/v, about 0.1% v/v to about 1% v/v, about 0.5% v/v to about 5% v/v, about 1% v/v to about 5% v/v, about 1.5% v/v to about 50% v/v, about 2% v/v to about 5% v/v, about 2.5% v/v to about 5% v/v, or a value between any two of these ranges. In some embodiments, the alkyl sulfate is in an amount of about 5% v/v, about 4% v/v, about 3% v/v, about 2.5% v/v, about 2% v/v, about 1.5% v/v, about 1% v/v, about 0.5% v/v, about 0.1% v/v, about 0.05% v/v, about 0.01% v/v, or a range between any two of these values.

In some embodiments, the composition may further include a solubilizer. In some embodiments, the solubilizer is biodegradable. In some embodiments, the solubilizer may be selected from esters of vegetable oils, glycerin, caprylic/capric glucoside, ethyl alcohol (grain), fatty alcohol, denatured alcohol, polyethylene glycol (PEG) dimethicone, almond oil polyglycerol, polysorbate 20, polysorbate 80, PEG-6 Caprylic/Capric Glycerides and Polyglycerol-6 Dioleate and Glyceryl Caprylate/Caprate such as that sold under the tradename Caprol Micro Express™, Di-PPG-2 Myreth-10 Adipate, coco glucoside, and heptyl glucoside. In some embodiments, the solubilizer is in an amount of about 0.01% v/v to about 5% v/v, about 0.1% v/v to about 5% v/v, about 0.1% v/v to about 4% v/v, about 0.1% v/v to about 3% v/v, about 0.1% v/v to about 2.5% v/v, about 0.1% v/v to about 2% v/v, about 0.1% v/v to about 1% v/v, about 0.5% v/v to about 5% v/v, about 1% v/v to about 5% v/v, about 1.5% v/v to about 50% v/v, about 2% v/v to about 5% v/v, about 2.5% v/v to about 5% v/v, or a value between any two of these ranges. In some embodiments, the solubilizer is in an amount of about 5% v/v, about 4% v/v, about 3% v/v, about 2.5% v/v, about 2% v/v, about 1.5% v/v, about 1% v/v, about 0.5% v/v, about 0.1% v/v, about 0.05% v/v, about 0.01% v/v, or a range between any two of these values.

In some embodiments, the composition may further include fragrances, preservatives, water, or a combination thereof.

Exemplary benefits of the adhesive remover of embodiments herein include moisturization of skin and/or eyelashes to which it is applied, longer preservation of the cosmetic item (e.g. false eyelashes), that it works on many brands of adhesives, and faster removal of cosmetic item/adhesive than comparable products on the market.

Some embodiments are directed to methods of removing an adhesive comprising applying onto the adhesive a composition comprising an alkyl sulfate or a salt thereof, and an essential oil. Some embodiments are directed to methods of removing an adhesive comprising applying onto the adhesive a composition comprising an alkyl sulfate or a salt thereof, and melaleuca oil. In some embodiments, the adhesive comprises an acrylate, an acrylamide, a cellulose, or a combination thereof. Some embodiments are directed to a method of removing false eyelashes comprising applying to the false eyelashes a composition comprising an alkyl sulfate or a salt thereof, and an essential oil. Some embodiments are directed to a method of removing false eyelashes comprising applying to the false eyelashes a composition comprising an alkyl sulfate or a salt thereof, and melaleuca oil. Some embodiments are directed to a method of removing a cosmetic item adhered to a body part comprising applying to the body part a composition comprising an alkyl sulfate or a salt thereof, and an essential oil. Some embodiments are directed to a method of removing a cosmetic item adhered to a body part comprising applying to the body part a composition comprising an alkyl sulfate or a salt thereof, and melaleuca oil.

The composition may be applied to the body part, cosmetic item, and/or the adhesive as a spray, a solution, a gel, a foam, or the like. The composition may be applied using an applicator, such as a cotton ball, cotton or foam swab, a foam wedge, a wipe, or the like.

Some embodiments are directed to a kit comprising an adhesive remover comprising an essential oil and an alkyl sulfate or a salt thereof; and instructions for use in methods for removing an adhesive. Some embodiments are directed to a kit comprising an adhesive remover comprising an essential oil and an alkyl sulfate or a salt thereof; and instructions for use in methods for removing a cosmetic item adhered to a body part. Some embodiments are directed to a kit comprising a false eyelash remover comprising an essential oil and an alkyl sulfate or a salt thereof; and instructions for use in methods for removing a false eyelash. In some embodiments, the kit may further include a cosmetic item to be adhered onto the body, glue, or a combination thereof In some embodiments, the cosmetic item to be adhered onto the body is false eyelashes.

This invention and embodiments illustrating the method and materials used may be further understood by reference to the following non-limiting examples.

### EXAMPLE 1

An eyelash adhesive remover was made by putting 1 mL of pure melaleuca oil into a 10 ml bottle, to which was added 1 mL of a surfactant, melaleuca oil, and solubilizer blend sold under the trade name Tough & Tender™. The bottle was then filled up with distilled water to make the final 10 mL composition.

### EXAMPLE 2

The effectiveness of the eyelash adhesive remover of Example 1 above was compared to the pure melaleuca oil and the surfactant, melaleuca oil, and solubilizer blend sold under the trade name Tough & Tender™.

A thick amount of an eyelash adhesive sold under the tradename Duo™ Eyelash Adhesive was applied to each of three mink lashes. The adhesive was allowed to dry for two hours and each lash was then cut in half. Each lash piece was placed into a shot glass. Lash pieces 1, 2, and 3 then received 14 sprays of one of the three test compositions, making sure the lash bands were fully submerged:

TABLE 1
TEST FORMULATIONS

| Lash | Formulation |
|---|---|
| 1 | 10 ml bottle with melaleuca oil, Tough & Tender ™, |
| | distilled water mix as in Example 1 |
| 2 | 10 ml bottle with 1 mL of melaleuca oil and then filled |
| | up with distilled water |
| 3 | 10 ml bottle with 1 mL of Tough & Tender ™ and then |
| | filled up with distilled water |

After two minutes, each lash piece was removed from the test solutions using tweezers and laid out to dry on a cookie sheet. Each lash piece was also patted dry with a paper towel.

Using tweezers, the adhesive was removed from each lash piece. The adhesive came off extremely easily with the formulation of Example 1. There was almost no resistance—the adhesive slid off without much assistance from the tweezers. There was no adhesive on the band of the lash and almost no force was used.

With the melaleuca oil mix, more force was needed to pull off the adhesive. While the adhesive did come off, it did need some use of the tweezers to punch the adhesive and pull off. There was no adhesive left on the lash band.

With the Tough & Tender™ mix, again, more force (similar to the force needed with the melaleuca oil) was needed to pull off the adhesive. While the adhesive did come off, it did need some use of the tweezers to remove the adhesive from the edges of the lash. There was no adhesive left on the band.

The experiment was repeated with the next set of lashes in new glass bottles. The results were repeated. All 3 solutions again successfully removed the adhesive from the lash bands but less force was needed from the tweezers with the solution of Example 1.

Conclusion: While the Tough & Tender™, a mixture of surfactant, melaleuca oil and solubilizer, as well as the melaleuca oil alone worked to remove the adhesive from the lash piece, the formulation of Example 1 was the most efficient and effortless at removing the adhesive, requiring less force and use of the tweezers.

Although the present invention has been described in considerable detail with reference to certain preferred embodiments thereof, other versions are possible. Therefore, the spirit and scope of the appended claims should not be limited to the description and the preferred versions contained within this specification.

1. A adhesive remover comprising an essential oil and an alkyl sulfate or a salt thereof.

2. The adhesive remover of claim 1, wherein the essential oil is melaleuca oil, eucalyptus oil, Eucalyptus giobuius oil, peppermint oil, lime oil, citrus oil, juniper oil, almond oil, anise oil, buchu oil, celery oil, cumin oil, nutmeg oil, cassia oil, cinnamon oil, sassafras oil, camphor oil, cedar oil, rosewood oil, sandalwood oil, agarwood oil, galangal oil, ginger oil, basil oil, bay leaf oil, cinnamon oil, sage oil, guava oil, lemongrass oil, oregano oil, patchouli oil, pine oil, rosemary oil, spearmint oil, thyme oil, tsuga oil, wintergreen oil, benzoin oil, copaiba oil, frankincense oil, myrrh oil, cannabis oil, chamomile oil, clary sage oil, clove oil, geranium oil, or any combination thereof.

3. The adhesive remover of claim 1, wherein the alkyl sulfate or a salt thereof is a palm-derived alkyl sulfate, a coconut-derived alkyl sulfate, or a combination thereof.

4. The adhesive remover of claim 1, wherein the alkyl sulfate is sodium coco sulfate, ammonium lauryl sulfate (ALS), sodium pareth sulfate, sodium lauryl sulfate, sodium laureth sulfate, or a combination thereof.

5. The adhesive remover of claim 1, wherein the essential oil is in the composition in an amount of about 2.5% v/v to about 20% v/v.

6. The adhesive remover of claim 1, wherein the alkyl sulfate is in an amount of about 0.01% v/v to about 5% v/v.

7. The adhesive remover of claim 1 further comprising a solubilizer, a fragrance, a preservative, or a combination thereof.

8. A method of removing an adhesive comprising applying onto the adhesive a composition comprising an alkyl sulfate or a salt thereof, and an essential oil.

9. A method of removing an adhesive comprising applying onto the adhesive a composition comprising an alkyl sulfate or a salt thereof, and melaleuca oil.

10. A method of removing false eyelashes comprising applying to the false eyelashes a composition comprising an alkyl sulfate or a salt thereof, and an essential oil.

11. A method of removing a cosmetic item adhered to a body part comprising applying to the body part a composition comprising an alkyl sulfate or a salt thereof, and an essential oil.

12. The method of any of claims 8-11, wherein the adhesive comprises an acrylate, an acrylamide, a cellulose, or a combination thereof.

13. A kit comprising an adhesive remover comprising an essential oil and an alkyl sulfate or a salt thereof; and instructions for use in methods for removing an adhesive.

14. The kit of claim 13 further comprising the adhesive.

15. A kit comprising an adhesive remover comprising an essential oil and an alkyl sulfate or a salt thereof; and instructions for use in methods for removing a cosmetic item adhered to a body part.

16. The kit of claim 15 further comprising the cosmetic item, an adhesive, or a combination thereof.

17. A kit comprising a false eyelash remover comprising an essential oil and an alkyl sulfate or a salt thereof; and instructions for use in methods for removing a false eyelash.

18. The kit of claim 17 further comprising false eyelashes, an adhesive, or a combination thereof.
